(19) United States
(12) Patent Application Publication
(10) Pub. No.: US 2011/0028828 A1
(43) Pub. Date: Feb. 3, 2011

(54) T1 RHO MAGNETIC RESONANCE IMAGING FOR STAGING OF HEPATIC FIBROSIS

(76) Inventors: DANIA DAYE, PHILADELPHIA, PA (US); RAVINDER REDDY, GLADWYNE, PA (US); REBECCA WELLS, WYNNEWOOD, PA (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(21) Appl. No.: 12/846,920

(22) Filed: Jul. 30, 2010

Related U.S. Application Data
(60) Provisional application No. 61/230,692, filed on Aug. 1, 2009.

(51) Int. Cl. A61B 5/055

(52) U.S. Cl. 600/410

(57) ABSTRACT

Methods for diagnosis of fibrotic diseases, staging of fibrotic diseases and monitoring treatment of fibrosis. The presence of fibrotic tissue is detected. First, a T_{1ρ} relaxation time of tissue is determined using magnetic resonance imaging. The determined T_{1ρ} relaxation time is then compared to a baseline T_{1ρ} relaxation time indicative of healthy tissue, and the presence of fibrotic tissue is then determined based on results of said comparison step. To determine a stage of fibrosis, a T_{1ρ} relaxation time of tissue is determined and compared to one or more calibrated T_{1ρ} relaxation times indicative of one or more stages of fibrosis, and the stage of fibrosis is determined based on results of said comparison step. The proposed technology offers a non-invasive MRI technique based on T_{1ρ} contrast that is sensitive enough to detect small changes of ECM hepatic protein concentration and architecture in all stages of hepatic fibrosis.

### CROSS REFERENCE TO RELATED APPLICATIONS

This application claims the benefit of U.S. Provisional Application No. 61/230,392, filed Aug. 1, 2009, the entirety of which is incorporated herein by reference.

### BACKGROUND OF THE INVENTION

1. Field of Invention

The present invention is directed to a method of using T1 Rho magnetic resonance imaging to detect and monitor the progression of fibrotic diseases.

2. Brief Description of the Prior Art

Cirrhosis of the liver is currently a significant cause of death in the United States of America. Wolf, D. C., “Cirrhosis,” eMedicine 2008. Despite the prevalence of liver disease, the primary method for diagnosing and monitoring the progress of patients afflicted with liver cirrhosis is liver biopsy. Manning, D. S. and Afdhal, N. H., “Diagnosis and Quantitation of Fibrosis,” Gastroenterology 2008, 134(6), pp. 1670-81. Liver biopsies, however, are invasive, associated with pain, have high complication rates, and may lead to morbidity and, in rare cases, mortality. Thampanitchawong, P., and Piratvisuth, T., “Livery Biopsy: Complications and Risk Factors,” World J. Gastroenterol. 1999, 5(4), pp. 301-4. In addition, liver biopsies are inaccurate due to sampling variability (Talwalkar, J. A., et al., “Magnetic resonance imaging of hepatic fibrosis: Emerging Clinical Applications,” Hepatology 2008 47(1), pp. 332-42), poor reproducibility (Yin, M., et al., “Quantitative Assessment of Hepatic Fibrosis in an Animal Model with Magnetic Resonance Elastography,” Magn. Reson. Med. 2007 58(2), pp. 346-53), inter- and intra-observer variability (Regev, A., et al., “Sampling Error and Intraobserver Variation in Liver Biopsy in Patients with Chronic HCV Infection,” Am. J. Gastroenterol. 2002 97(10), pp. 2614-18) and the fact that liver biopsies typically sample only about 1/50,000 of the liver tissue. Liver biopsies have also been reported to misclassify up to one third of cirrhotic livers. Wells, R. G., “Antifibrotic and HapatoProtectant Therapies—Hot Prospect and Challenges to Clinical Testing,” In 2008 AASLD Postgraduate Course. These shortcomings impede the care of individual patients as well as the administration of clinical trials, and demonstrate that there is a need for a new, reliable and non-invasive clinical too for diagnosis and quantitative tracking of fibrotic liver disease.

U.S. Pat. No. 5,322,682 discloses a method for quantitatively measuring and mapping stored iron in tissue using magnetic resonance imaging (MRI). This patent employs the difference between a relaxation time T2 measured at a first magnetic field strength and a relaxation time T2 measured at a second magnetic field strength to generate a field dependent signal which is then correlated with the stored iron in the form of ferritin at the measured location of the patient. A visual two-dimensional display of the patient's iron stores can be generated from multiple measurements. This method can be used to monitor abnormal iron accumulation in the liver but is not used to diagnose or stage fibrosis.

A correlation between cirrhosis of the liver and hepatic venous morphology using non-invasive methods has been identified in Zhang, Y. et al., “Changes in Hepatic Venous Morphology with Cirrhosis on MRI,” Journal of Magnetic Resonance Imaging, Vol. 29, issue 5, pp. 1085-1092, published online on Apr. 22, 2009. MRI was used to identify changes in the venous morphology of patients with cirrhosis and this morphology was compared to liver donor candidates with healthy livers. It was concluded that small hepatic veins, minimally enlarged main portal vein and small intrahepatic portal veins may facilitate identification of cirrhosis using MRI.

Another non-invasive technique for examination of hepatic cirrhosis is disclosed in WO 01/25785 A1. In this method, respiratory expiration is collected from a patient and the isopropanol and/or cyanide compounds in the expiration and quantified and correlated with liver disease.

Other known techniques for diagnosis and/or monitoring of fibrotic disease include Fibroscan®, one embodiment of which is described in WO 2004/016176, FibroMAX® and FibroSURE™ described in U.S. Pat. No. 6,631,330. Although magnetic resonance elastography (MRE) has shown promising in accurately classifying liver fibrosis based on liver stiffness, it was finally shown to be unable to distinguish between the early stages of fibrosis. MRE assumed a linear relationship between fibrosis and liver stiffness. More recent studies, however, have shown that this is not necessarily the case during early stages of fibrosis. Bateller, R. and Brenner, D. A., “Liver Fibrosis,” J. Clin. Invest. 2005 115(2), pp. 209-218 and Georges P. C. et al. “Increased Stiffness of the Rat Liver Precedes Matrix Deposition: Implications for Fibrosis,” Am. J. Physiol. Gastrointest. Liver Physiol. 2007; 293(6):G1147-54. These techniques above are all able to differentiate between mild and severe fibrosis, but are unable to make finer distinctions. As a result, it is clear that these techniques do not offer a method for accurately staging early or intermediate fibrosis or for monitoring the progress of patients over short time periods.

U.S. Patent application publication no. US 2003/0218459 A1 describes pulse imaging sequences and methods for two-dimensional multi-slice T_{1ρ}-weighted and three-dimensional T_{1ρ}-weighted MRI. Also provided is a self-compensating spin-locking sequence for correcting and reducing, artifacts in T_{1ρ}-weighted MRI and a sequence combining three dimensional T_{1ρ}-weighted MRI with a self-compensating spin-locking pulse for facilitating T_{1ρ}-weighted imaging with surface coils. This method can be employed to assess the amount of proteoglycans in a sample. In vivo imaging was carried out on the human knee joint, bovine patallae and human patellar cartilage for imaging patellar cartilage and quantifying the degree of abnormality in the cartilage.

WO 2008/048641 A2 discloses a method and system for rapid MRI scanning for measurement of T_{1ρ} relaxation times for the preparation of two- and three-dimensional T_{1ρ} maps for visualization of anatomical structures. The scanning methodology is disclosed as being useful for studying pathology such as cartilage pathology and arthritis, intervertebral disk pathology and lower back pain, tumors, Alzheimer's disease and neural degeneration and myocardial abnormality and heart disease.

Hepatic fibrosis and cirrhosis, the common end result of chronic liver injury, is characterized by the formation of irregular broad bands of fibrous tissue (fibrosis). It is a highly morbid and potentially fatal condition. Multiple different non-invasive approaches have been developed for diagnosis and monitoring of hepatic fibrosis, however none are able to achieve sufficient sensitivity to accurately stage fibrosis, especially in its earliest stages. Specifically, serum markers, ultrasound, T1- and T2-weighted MRI, and contrast-enhanced MRI were not able to differentiate the early and intermediate stages of fibrosis or could only detect late-stage fibrosis; they are unable to detect small differences from one time to the next in individual patients. Similarly, studies using diffusion-weighted MR imaging and MR spectroscopy showed significant discrepancies and low sensitivities in staging the early and middle stages of fibrosis. MR elastography can more accurately stage liver fibrosis based on stiffness, although it is ineffective in distinguishing between the early stages of fibrosis and is able to detect only significant differences in the amount of fibrosis.

Fibrosis is a dynamic process that progresses at dramatically different rates in different patients thereby necessitating the use of a diagnostic and monitoring technique with sufficient sensitivity to measure early and small changes in the state of hepatic extracellular matrix (ECM) proteins over short periods of time.

There remains a need for a reliable, non-invasive method for diagnosing and monitoring the progress of fibrotic diseases of the liver.

### SUMMARY OF THE INVENTION

In a first aspect, the present invention provides a method for diagnosis of fibrotic diseases. In the method, the presence of fibrotic tissue is detected. First, a T_{1ρ} relaxation time of tissue is determined using magnetic resonance imaging. The determined T_{1ρ} relaxation time is then compared to a baseline T_{1ρ} relaxation time indicative of healthy tissue, and the presence of fibrotic tissue is then determined based on results of said comparison step.

In a second aspect, the present invention provides a method for determining a stage of fibrosis. In this method, a T_{1ρ} relaxation time of tissue is determined using magnetic resonance imaging. The determined T_{1ρ} relaxation time is then compared to one or more calibrated T_{1ρ} relaxation times indicative of one or more stages of fibrosis, and the stage of fibrosis is determined based on results of said comparison step.

In a third aspect, the present invention provides a method of monitoring progress of a therapeutic treatment of fibrosis. In this method, a baseline value of a T_{1ρ} relaxation time of fibrotic tissue is measured prior to or upon initiation of said treatment using magnetic resonance imaging. One or more additional T_{1ρ} relaxation times are then measured after initiation of the treatment. At least one of the one or more T_{1ρ} relaxation times measured after initiation of the treatment is compared to the baseline value of the T_{1ρ} relaxation time and an effectiveness of the treatment is determined based on results of said comparison step. Additional T_{1ρ} relaxation times measured after initiation of the treatment may also be used as baseline values for comparison to subsequently measured T_{1ρ} relaxation times to continue to monitor the treatment as the treatment progresses.

The proposed technology offers a non-invasive MRI technique based on T_{1ρ} contrast that can be sensitive enough to detect small changes in ECM protein concentration and architecture in all stages of hepatic fibrosis.

### BRIEF DESCRIPTION OF THE DRAWINGS

FIG. 1 shows the dependence of R_{1ρ} dispersion on chondroitin sulfate concentration.

FIG. 1A shows the dispersion of the buffer (triangles) is less than that of 2% (diamonds), 5% (circles) and 10% (squares) solutions of chondroitin sulfate. The correlation time of these dispersion plots is in agreement with literature values for hydroxyl exchange times under similar conditions.

FIG. 1B shows the dependence of R_{1ρ} with chondroitin sulfate concentration at various spin-lock amplitudes: 314 rad/s (circles), 930 rad/s (squares), 4,650 rad/s (triangles) and 1.1×10⁴ rad/s (diamonds).

FIG. 2 shows a T_{1ρ} map of collagen phantoms prepared by the method of Example 1. The collagen concentrations, listed counter-clockwise from the top, were control (no collagen), 3.25 mg/ml, 7.5 mg/ml, 10 mg/ml, 15 mg/ml, 25 mg/ml and 30 mg/ml.

FIG. 3 shows the linear correlation between the T_{1ρ} relaxation and collagen concentration as measured in Example 1.

FIG. 4 shows that increasing the amount of collagen present in the samples of Example 1 is associated with changes in the T_{1ρ} dispersion thereby indicating that increased collagen levels can be correlated with T_{1ρ} dispersion for measurement purposes.

FIG. 5 shows measurements of T_{1ρ} relaxation time for a series of liver explants for Example 2. The T_{1ρ} relaxation time increases with the degree of fibrosis progression.

FIG. 6 is a plot of the T_{1ρ} relaxation time versus the progression of liver fibrosis. This plot shows that longer T_{1ρ} relaxation times are seen with greater progression of the liver fibrosis.

FIG. 7 is a T_{1ρ} map of an axial image of a human volunteer was performed at the thoracic level using a T_{1ρ} imaging sequence at different spin lock amplitudes.

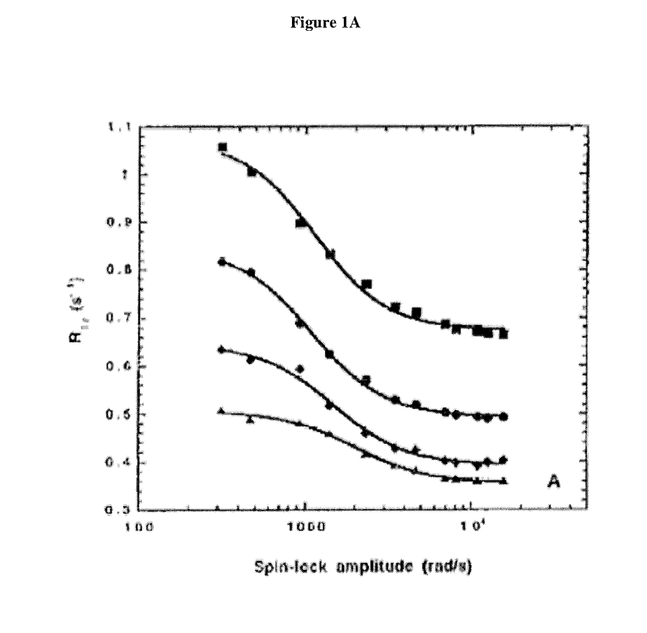

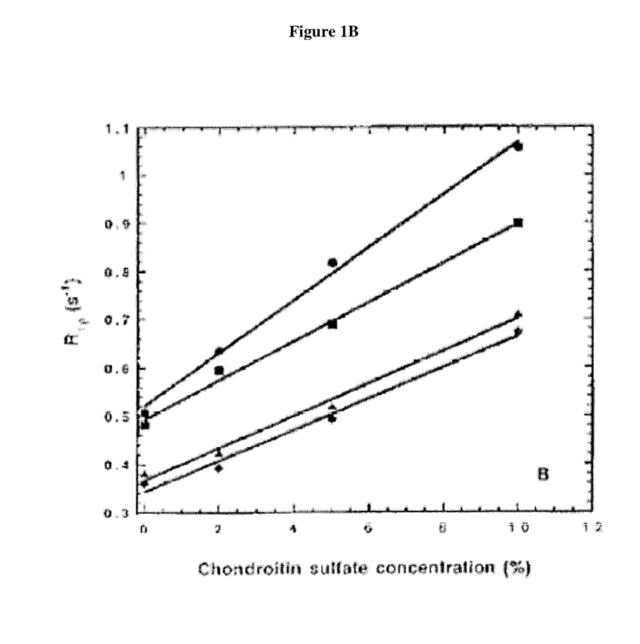

### DETAILED DESCRIPTION OF THE PREFERRED EMBODIMENTS

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

It must be noted that as used herein and in the appended claims, the singular forms “a”, “an”, and “the” include plural references unless the context clearly dictates otherwise. As well, the terms “a” (or “an”), “one or more” and “at least one” can be used interchangeably herein. It is also to be noted that the terms “comprising”, “including”, and “having” can be used interchangeably.

Hepatic fibrosis is characterized by progressive deposition of, and the reorganization of, extracellular matrix (ECM) proteins in the liver. Early diagnosis and regular monitoring of liver fibrosis is especially important, as this would allow for the initiation of anti-fibrotic therapies capable of halting or even reversing this process. (Bhat, V. and Bhat, M., MJM, 2008 11(1) p. 39) This technology is uniquely capable of doing this as it can be applied multiple times, is sensitive enough to assess early stage fibrosis, and can detect small changes in the amount of ECM proteins in the liver.

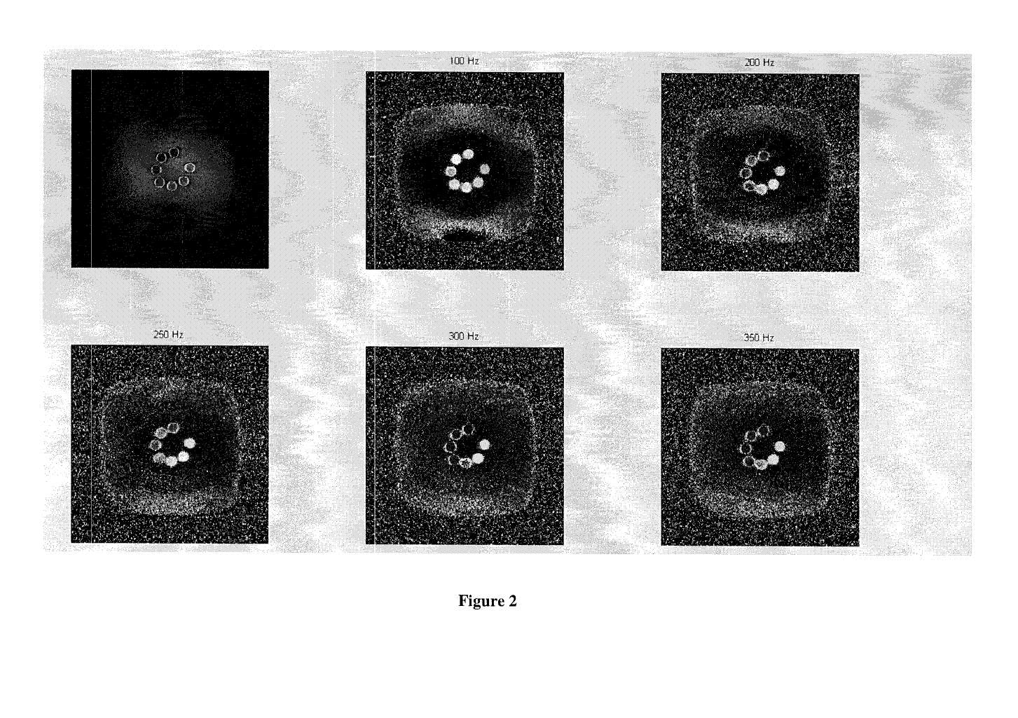

Described is a new technique that uses MRI to measure the T_{1ρ} relaxation exponential decay time constant of excited nuclear spin magnetization during a constant amplitude “spin-lock” radiofrequency pulse. Duvvuri, U., et al., “Water Magnetic Relaxation Dispersion in Biological Systems: the Contribution of Proton Exchange and Implications for the Noninvasive Detection of Cartilage Degradation,” Proc. Natl. Acad. Sci. USA 2001 98(22), pp. 12479-84. The T_{1ρ} relaxation rate is sensitive to chemical exchange between water protons and the —OH and —NH groups on macromolecules when the exchange rate occurs at frequencies close to the spin-lock frequency. Wheaton, A. J., et al., “Quantification of Cartilage Biomechanical and Biochemical Properties via T_{1ρ} Magnetic Resonance Imaging,” Magn. Reson. Med. 2005, 54(5), pp. 1087-93. Also, the dipolar coupling interactions contribute to T_{1ρ} signals at low spin-lock amplitude. These relationships are illustrated in FIG. 2 herein. The T_{1ρ} relaxation rate increases with increasing macromolecular content.

The T_{1ρ} relaxation of water molecules is linearly correlated to protein concentration, especially proteoglycans. Based on this principle, this technique has been previously used as a surrogate index for biochemical and biomechanical changes in human articular cartilage. In vivo studies in humans and animal models correlated T_{1ρ} to collagen concentration and architecture in articular cartilage. Wheaton, A. J., et al., “Quantification of Cartilage Biomechanical and Biochemical Properties via T_{1ρ} Magnetic Resonance Imaging,” Magn. Reson. Med. 2005, 54(5), pp. 1087-93.

Since liver fibrosis is a progressive deposition and reorganization of ECM protein macromolecules, the residual static dipolar coupling and chemical exchange between water protons and protons on the ECM protein macromolecules will provide a T_{1ρ} concentration-dependent response as well as an architecture dependent response due to the presence of the residual static dipolar coupling. Since increasing fibrosis of the liver is accompanied by increasing water content, T_{1ρ} relaxation times will increase with increasing fibrosis.

The present invention uses this MRI technique to measure increased deposition of common ECM components, mainly Type I and III collagens (the main ECM components whose concentration have been shown to increase in fibrosis), in the liver to provide an indication of the type of and/or progression of liver fibrosis. Furthermore, because dipolar coupling interactions contribute to T_{1ρ} signals at low spin-lock amplitude, the magic angle effect can be used to determine the architecture of ECM proteins, and thus further characterize the stage of liver fibrosis, especially during earlier stages that precede increased ECM deposition.

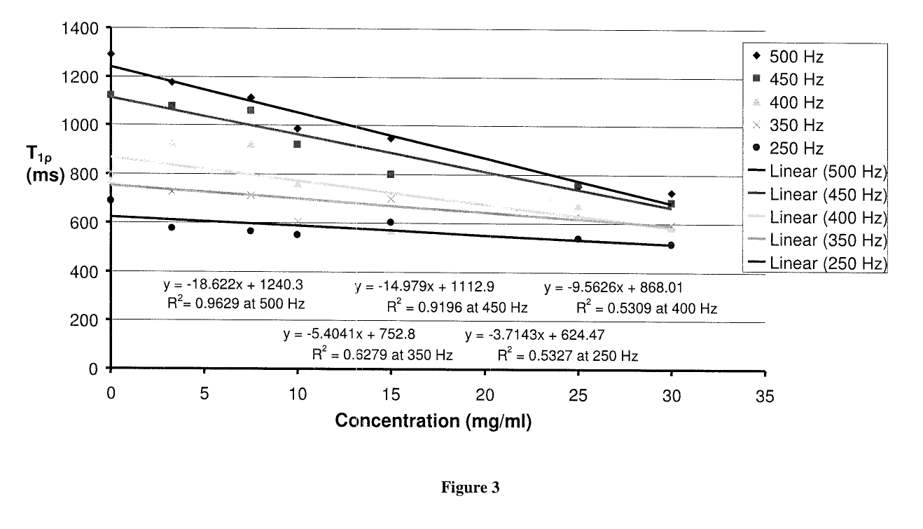

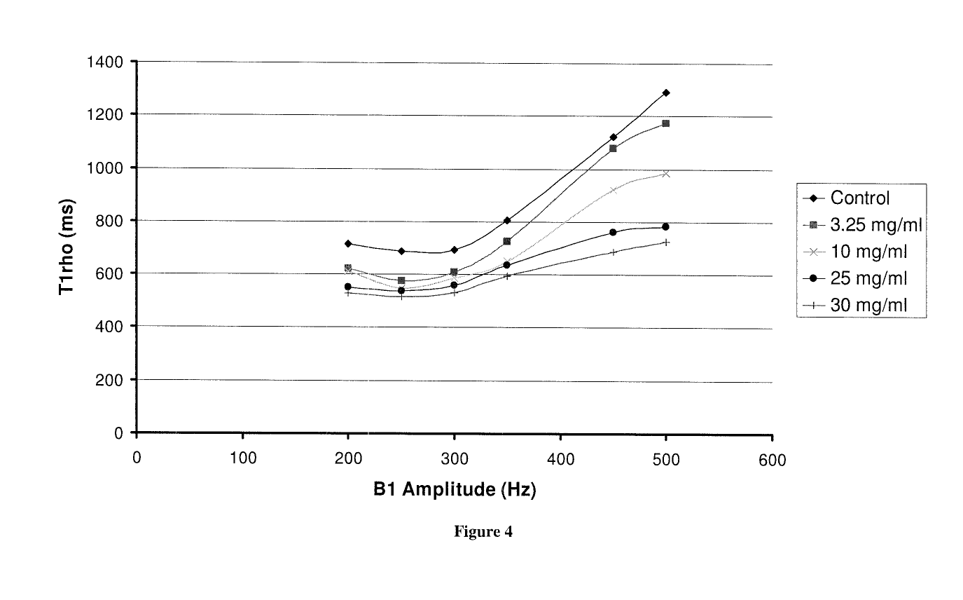

T_{1ρ} MR Imaging can be used to detect the precursors of fibrosis. More specifically, the change in architecture of or increased deposition of ECM components that will form into cirrhosis or other fibrotic disease can be detected at an early stage using T_{1ρ} MR Imaging. T_{1ρ} relaxation time is linearly correlated to the concentration of collagen and other ECM components (i.e. proton exchanging species). For example, in Example 1 below it is shown that T_{1ρ} relaxation times correlate with different concentrations of collagen I (see e.g. FIGS. 2-3). FIG. 4 also shows that increasing the amount of collagen present in the samples is associated with changes in the T_{1ρ} dispersion. This provides a further indication that increased collagen levels can be correlated with T_{1ρ} dispersion. This demonstrates that it is possible to detect liver fibrosis using the T_{1ρ} MR imaging technique.

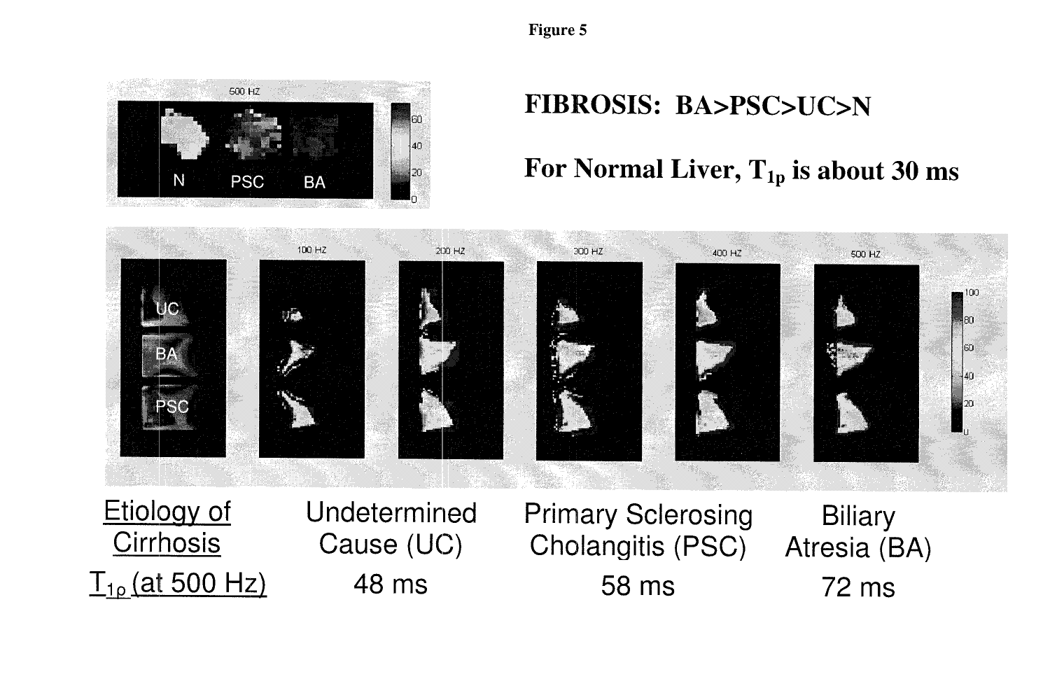

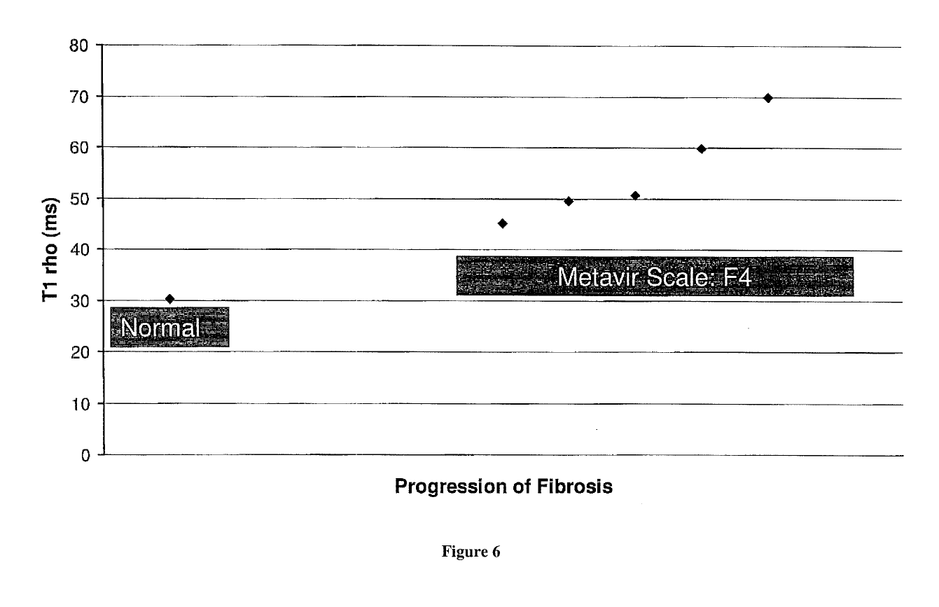

In Example 2, T_{1ρ} relaxation times were measured for various liver explants. FIGS. 5-6 show that T_{1ρ} relaxation time can be correlated with the degree of fibrosis progression. This demonstrates that it is possible to monitor the progression of liver fibrosis using the T_{1ρ} MR imaging technique.

Also, T_{1ρ} MR imaging can be used to determine the architecture of ECM proteins since the dipolar coupling interactions contribute to T_{1ρ} signals at low spin-lock amplitude. As a result, the magic angle effect can be used to determine the architecture of ECM proteins in fibrosis. In MRI, the magic angle is a precisely defined angle at which any two nuclei with a coupling vector oriented at an angle of 54.7 degrees relative to the external magnetic field of the MRI, have zero dipolar coupling. When the nuclei of the macromolecules are oriented along the magic angle, the contribution from the dipolar interactions is zero leading to a reduction in the total T_{1ρ} signal. By detecting and quantifying the reduction in this signal, the architectural changes in the extracellular matrix in the early stages of fibrosis can be characterized. This technique has been previously used to detect and quantify the orientation of macromolecules in different layers of articular cartilage in the knee (Borthakur, E. Mellon, S. Niyogi, W. Witschey, J. B. Kneeland, R. Reddy, Sodium and T₁ᵣₕₒ MRI for molecular and diagnostic imaging of articular cartilage, NMR Biomed 19(7), 781-821 (2006)). The concentration and architecture of the ECM proteins, taken together, can be used to determine the extent of fibrosis. At present, the gold standard clinical method to stage hepatic fibrosis is the liver biopsy, based on pathological staging schemes, such as the Metavir scale. These schemes stage fibrosis based on the architecture of the collagen deposition (and to a lesser extent the amount of collagen deposition) which can be detected in the liver biopsies. The Metavir scale, for instance, consists of 5 stages, F0=no fibrosis (normal liver), F1=portal fibrosis or mild fibrosis, F2=few septa or moderate fibrosis, F3=many septa or severe fibrosis and F4=cirrhosis. Those stages are currently used as the basis for determining when treatment is indicated, e.g. for patients with a Metavir stage greater than or equal to 2, and are also used to predict patient prognosis.

In contrast, the technique of the present invention has the unique ability to both (1) identify the architecture and (2) quantify the amount of collagen and ECM component deposition in the liver. This will provide a significant improvement in the accuracy of staging the extent of fibrosis through the identification of more subtle changes than what is currently possible using a liver biopsy. The technique of the present invention has sufficient sensitivity to accurately stage early fibrosis as well as monitor the progress of patients over short periods of time by detecting smaller changes in the patient's condition than is currently possibly using the liver biopsy method. At present, there are no approved antifibrotic therapies available and the lack of accurate and safe tools to diagnose fibrosis is a major impediment to conducting clinical trials for promising new therapies.

It has been suggested that in the development of liver fibrosis, early changes are characterized by architectural changes, specifically collagen cross-linking, which tends to orient the collagen fibers linearly in a specified direction. This change in architecture, seen in the earlier stages of fibrosis (Metavir F1), can be quantified using the present invention by application of the magic angle effect as discussed above.

Following the initial architectural changes, subsequent stages of fibrosis are typically characterized by increased matrix deposition of ECM components (Metavir F2-F4). The increased ECM deposition which characterizes these stages of fibrosis can be quantified using the T_{1ρ} relaxation time. By quantifying both the initial changes in architecture and the subsequent increased ECM deposition, the present technique provides a non-invasive and more accurate alternative to the Metavir staging system for staging liver fibrosis, which has already been shown to correlate with patient prognosis.

Spin-lock MRI utilizes low amplitude spin-lock radiofrequency pulses to generate image contrast. The T_{1ρ} relaxation time, describing the time of relaxation of magnetization under the influence of spin-locking, can be measured non-invasively to yield quantitative information about low frequency interactions between bulk water and surrounding molecules in biological tissues.

Suitable methods for performing spin-lock MRI are known to persons skilled in the art. For example, a method of performing spin-lock MRI suitable for use in the present invention is described in U.S. patent application publication no. US 2003/0218459 A1, the disclosure of which is hereby incorporated by reference for the purpose of describing a suitable spin-lock MRI method.

Another suitable method for performing spin-lock MRI for use in the present invention is described in WO 2008/04861 A2, the disclosure of which is hereby incorporated by reference for the purpose of describing a suitable spin-lock MRI method.

The present invention can be applied, for example, in patients at high risk for developing liver cirrhosis, especially those with viral hepatitis, alcoholism, metabolic syndrome, and certain autoimmune diseases. Furthermore, the present invention may be used as an indicator for prescribing anti-fibrotic therapies. The present invention may also provide a reliable, non-invasive clinical tool able to provide information for objective diagnosis and quantification of the stages of fibrosis leading to, for example, cirrhosis. This may provide the ability to quantitatively track liver diseases for the purpose of treatment assessment as well as use in clinical trials for development of new anti-fibrotic therapies.

Thus, in a method in accordance with one embodiment of the present invention, spin-lock MRI is used to diagnose liver fibrosis in a patient. In this method, the T_{1ρ} relaxation time is determined and compared to an expected T_{1ρ} relaxation time for healthy liver tissue, as shown, for example, in FIG. 5 of the present application. A variation in the T_{1ρ} relaxation time relative to that of healthy liver tissue provides an indication that the patient may be suffering from liver fibrosis. Thus, in the case of FIG. 5, the T_{1ρ} relaxation time for normal liver tissue using the particular apparatus described in the examples was calibrated as being between about 25 to 30 ms. Therefore, T_{1ρ} relaxation times in excess of about 30 ms observed using this apparatus would provide an indication that the patient may have liver fibrosis. It is expected that due to the sensitivity and reliability of the T_{1ρ} relaxation time measurements relative to collagen concentrations, that the present method will provide the ability to diagnose the presence of liver fibrosis even at very early stages of development, thereby allowing earlier, potentially more effective treatment of the liver fibrosis than using presently available, less sensitive and less reliable diagnostic methods for liver fibrosis.

In a method in accordance with another embodiment of the present invention, staging of liver fibrosis may be monitored. In this method, an initial baseline measurement T_{1ρ} relaxation time is taken to indicate the initial stage of liver fibrosis for a particular patient. Then, subsequent measurements of T_{1ρ} relaxation time may be taken at various time intervals and compared to the initial baseline measurement and/or to each other in order to monitor the progression of the liver fibrosis. For example, the data presented in FIG. 6 demonstrates that it is possible to accurately monitor progression of liver fibrosis using measurements of T_{1ρ} relaxation times. Thus, in the case of the data shown in FIG. 6, longer T_{1ρ} relaxation times correlate with further progression of the liver fibrosis in the patient. By comparing sequential measurements taken at various time intervals, the progression of the liver fibrosis can be monitored in this manner. This methodology may provide particularly useful as a method for monitoring the effectiveness of liver fibrosis treatments. This method can be used, for example, to monitor clinical trials of potential new treatments for liver fibrosis as well.

The method of the present invention is not limited to detection of liver fibrosis, but rather is potentially applicable for diagnosis and monitoring of diseases, ailments, disorders or other problems which are characterized by the development of fibrotic tissue. Another application of the method of the present invention is for screening for hypertrophic cardiomyopathy (HCM), the number one condition responsible for sudden death in young athletes. Recently, it has been shown that hearts afflicted by HCM exhibit fibrotic changes. At present, there is no reliable technique to screen young athletes for this potentially fatal condition. The method of the present invention can be modified to detect fibrosis in HCM and thus provide for a non-invasive tool for screening for this potentially fatal condition. Thus, the present invention may also be employed as a diagnostic tool or for monitoring the progression of fibrotic tissue at various locations in the body.

The following examples are provided to further illustrate embodiments of the present invention.

### EXAMPLES

### Example 1

T_{1ρ} magnetic resonance scans were performed on collagen phantoms having different concentrations of Type I collagen, the main protein that increases during fibrosis. A 3T Siemens Trio MRI scanner was used. 1 cm×1 cm×1 cm liver explant samples were placed in a 42-well plate and the plate was mounted on a custom-built coil and imaged. The images are shown in FIG. 2.

A linear correlation between T_{1ρ} relaxation time and collagen concentration was observed, as shown in FIG. 3. Relaxation times increased as collagen concentrations decreased as shown in FIG. 4.

### Example 2

In this example, T_{1ρ} magnetic resonance scans were performed on several different liver explants including normal liver tissue (N), liver tissue with cirrhosis of undetermined cause (UC), liver tissue with cirrhosis caused by primary sclerosing cholangitis (PSC) and liver tissue with cirrhosis caused by biliary atresia. The results are shown in FIG. 5. Normal liver tissue exhibited a T_{1ρ} relaxation time of about 25-30 ms, whereas the cirrhotic liver tissues each exhibited different T_{1ρ} relaxation times thereby showing that the T_{1ρ} relaxation time can differentiate between the degree of matrix abnormality even in cirrhotic livers that would be classified at the same stage (Metavir F4) by standard histological staging systems. FIG. 6 shows a plot of the T_{1ρ} relaxation time versus the progression of liver fibrosis showing that the more extensive the liver fibrosis the longer the T_{1ρ} relaxation time.

### Example 3

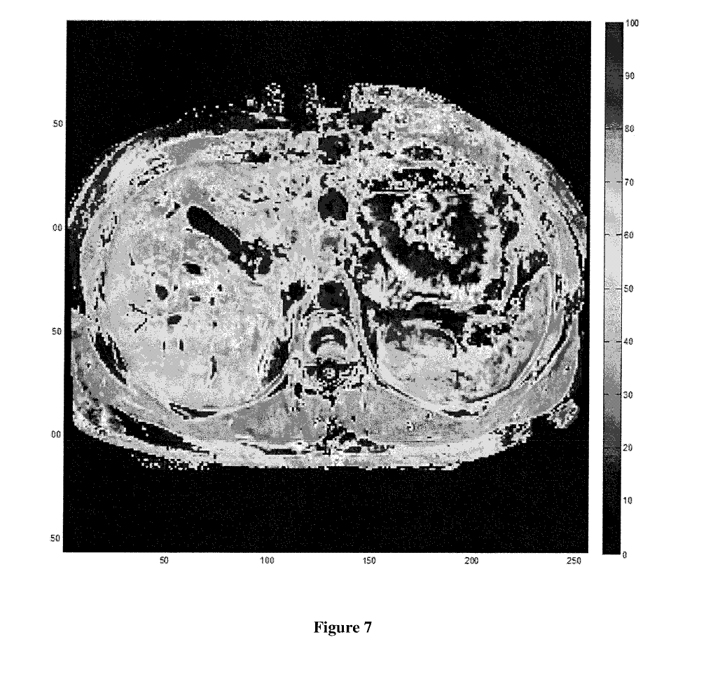

In this example, T_{1ρ} magnetic resonance scans were performed in vivo on a human liver. An axial image of a human volunteer was performed at the thoracic level using a T_{1ρ} imaging sequence at different spin lock amplitudes and a T_{1ρ} map was generated (FIG. 7). This study showed that the human liver has a heterogeneous distribution of relaxations over its surface with T_{1ρ} values ranging between 25 seconds and 50 seconds. This heterogeneity is most likely correlated with the normal micro-architecture of the liver. Regions of the liver with higher macromolecular content have higher relaxation times. In pathologic conditions, an increase in T_{1ρ} relaxation times in the affected areas is expected, as compared to normal tissue. This example shows the feasibility of the technique in humans.

From these examples it can be concluded that T_{1ρ} magnetic resonance scans can be employed to quantify changes in collagen concentrations and that the T_{1ρ} signal can be directly correlated with the histological stages of fibrosis. Thus, it is expected that T_{1ρ} magnetic resonance scans may provide a quantitative, non-invasive assessment of fibrosis in the liver.

The foregoing examples have been presented for the purpose of illustration and description and are not to be construed as limiting the scope of the invention in any way. The scope of the invention is to be determined from the claims appended hereto.

What is claimed is:

1. A method for detecting a presence of fibrotic tissue comprising the steps of:
determining a T_{1ρ} relaxation time of tissue using magnetic resonance imaging,
comparing the determined T_{1ρ} relaxation time to a baseline T_{1ρ} relaxation time indicative of healthy tissue, and
determining the presence of fibrotic tissue based on results of said comparison step.

2. A method as claimed in claim 1 wherein the presence of fibrotic tissue is determined from measurement of a T_{1ρ} relaxation time longer than the baseline T_{1ρ} relaxation time.

3. A method as claimed in claim 2, wherein the tissue is liver tissue.

4. A method as claimed in claim 3, wherein the presence of fibrotic tissue indicates liver fibrosis.

5. A method as claimed in claim 3, wherein the presence of fibrotic tissue indicates liver cirrhosis.

6. A method as claimed in claim 1, further comprising the step of determining a stage of fibrosis by comparing the determined T_{1ρ} relaxation time to one or more calibrated T_{1ρ} relaxation times indicative of one or more stages of fibrosis.

7. A method as claimed in claim 6, wherein the step of determining a stage of fibrosis further comprises the step of determining the concentration and architecture of matrix proteins of said fibrotic tissue and comparing the determined concentration and organization to one or more calibrated concentrations and architectures indicative of one or more stages of fibrosis.

8. A method for determining a stage of fibrosis comprising the steps of:
determining a T_{1ρ} relaxation time of tissue using magnetic resonance imaging,
comparing the determined T_{1ρ} relaxation time to one or more calibrated T_{1ρ} relaxation times indicative of one or more stages of fibrosis, and
determining the stage of fibrosis based on results of said comparison step.

9. A method as claimed in claim 8 wherein the stage of fibrosis is determined from measurement of a T_{1ρ} relaxation time that falls within a predetermined range of T_{1ρ} relaxation times indicative of a particular stage of fibrosis.

10. A method as claimed in claim 9, wherein the tissue is liver tissue.

11. A method as claimed in claim 9, wherein the fibrosis is liver fibrosis.

12. A method as claimed in claim 9, wherein the fibrosis is liver cirrhosis.

13. A method as claimed in claim 9, wherein the step of determining a stage of fibrosis further comprises the step of determining the concentrations and architecture of said fibrotic tissue and comparing the determined concentrations and architecture to one or more calibrated concentrations and architecture indicative of one or more stages of fibrosis.

14. A method of monitoring progress of a therapeutic treatment of fibrosis comprising the steps of:
measuring a baseline value of a T_{1ρ} relaxation time of fibrotic tissue prior to or upon initiation of said treatment using magnetic resonance imaging,
measuring one or more additional T_{1ρ} relaxation times after initiation of said treatment,
comparing at least one of the one or more additional T_{1ρ} relaxation times measured after initiation of the treatment to the baseline value of the T_{1ρ} relaxation time, and
determining an effectiveness of the treatment based on results of said comparison step.

15. A method as claimed in claim 14, further comprising the step of comparing at least one of the one or more T_{1ρ} relaxation times measured after initiation of said treatment to at least one other of the one or more T_{1ρ} relaxation times measured after initiation of said treatment, and
determining an effectiveness of the treatment based on results of said step of comparing of the at least two T_{1ρ} relaxation times measured after initiation of said treatment.

16. A method as claimed in claim 14 wherein measurement of a T_{1ρ} relaxation time that falls below the baseline T_{1ρ} relaxation time is indicative of a beneficial treatment result.

17. A method as claimed in claim 15, wherein measurement of a T_{1ρ} relaxation time that falls below a T_{1ρ} relaxation time measured earlier in time is indicative of a beneficial treatment result.

18. A method as claimed in claim 15, wherein the tissue is liver tissue.

19. A method as claimed in claim 15, wherein the fibrosis is liver cirrhosis.
